(19) United States
(12) Patent Application Publication
(10) Pub. No.: US 2011/0109995 A1
(43) Pub. Date: May 12, 2011

(54) BEARING LUBRICANT, BEARING AND DISK DRIVE DEVICE

(75) Inventors: Mitsuo KODAMA, Shizuoka (JP); Takuji YAMADA, Shizuoka (JP); Hiroyuki ANZAI, Hyogo (JP)

(73) Assignees: ALPHANA TECHNOLOGY CO., LTD., Shizuoka (JP); BALBIS CO., LTD., Hyogo (JP)

(21) Appl. No.: 12/616,110

(22) Filed: Nov. 10, 2009

(51) Int. Cl. G11B 5/60; C07C 69/34; C10M 105/36; C10M 137/04

(52) U.S. Cl. 360/234.1; 560/190; 508/496; 508/440; 508/100; G9B/5.229

(57) ABSTRACT

A bearing lubricant includes a base oil containing an ester compound consisting of 2-ethyl-2-methyl-1,3-propanediol and at least one of carboxylic acids having 5 to 12 carbon atoms, the ester compound being represented by the following formula (1), in which the base oil has a kinetic viscosity at approximately 40° C. of approximately 7 to 15 mPa, and has a pour point of approximately −60° C. or less:

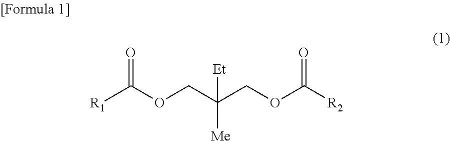

where R1 and R2 represent hydrocarbon groups having 4 to 11 carbon atoms.

### BACKGROUND OF THE INVENTION

1. Field of the Invention

The present invention relates to a bearing lubricant, a bearing using the lubricant, and a disk drive device provided with the bearing.

2. Description of the Related Art

Electronic apparatuses such as personal computers and mobile recording devices have been smaller in size and less power consuming year after year. Also, temperature ranges in which these electronic apparatuses can be used have been wider year after year. With these advancements, spindle motors mounted with fluid dynamic bearings or impregnated bearings, etc., have been used in disk drive devices using a small high-speed rotating recording medium, for example, a magnetic disk, the disk drive devices being used in these electronic apparatuses. These electronic apparatuses can be smaller in size, less power consuming and used in enlarged temperature ranges, due to the performance improvement of motors.

Under these situations, there have been increasing demands in recent years that the electronic apparatuses should process at higher-speeds, be smaller in size, be less power consuming, have longer life spans, or be used in enlarged temperature ranges. On the other hand, there has been a need that apparatuses mounted with disk drive devices should be widely used in mobile apparatuses, etc., and hence motors and apparatuses mounted with motors have been required to withstand the uses under more severe environments and to have longer life spans. That is, motors and apparatus mounted with motors are strongly required to be used in enlarged temperature ranges and to have longer life spans.

One of the measures to realize the performance improvement of a motor and an apparatus mounted with a motor, is to enhance the performance of a bearing such as a fluid dynamic bearing, etc., mounted in the motor. To realize the performance improvement of a bearing, Japanese Patent Application Publication No. 2006-83321, International Patent Publication Pamphlet No. 2004/018595 and Japanese Patent Application Publication No. 2008-7741, disclose various bearing lubricants.

To improve the performance of a bearing, it can be considered that an evaporation loss of a bearing lubricant to be used is reduced such that the bearing lubricant has a long life span. Generally, in order to reduce the evaporation loss of a bearing lubricant, the molecular weight of a base oil contained in the bearing lubricant is required to be large. However, if the molecular weight thereof is made large, the viscosity of the bearing lubricant is increased, thereby an energy loss in the bearing becomes large, resulting in an increase in power consumption. In contrast, if the molecular weight of the base oil is made small in order to reduce the energy loss in a bearing by making the viscosity of the bearing lubricant small, the evaporation loss of the bearing lubricant is increased. Also, because the life span of a fluid dynamic bearing is dependent on an amount of the bearing lubricant held inside, the increase in the evaporation loss of the bearing lubricant drastically shortens the life span of the fluid dynamic bearing.

Further, a disk drive device provided with a fluid dynamic bearing is structured with an extremely high accuracy. Accordingly, it is almost impossible to repair the bearing mounted inside a disk drive device. Due to this, the disk drive device in which the bearing reaches the end of its life span has to be discarded such that the device is replaced by a new one, even when parts other than the bearing can be sufficiently used. As stated above, a situation in which other parts besides a bearing have to be discarded due to the single bearing at the end of its life span, is not desirable in terms of an environmental resource issue.

The stiffness of a fluid dynamic bearing (hereinafter, sometimes and appropriately referred to bearing stiffness) depends on the viscosity of a bearing lubricant. Accordingly, there has been a problem that, when the molecular weight of a base oil is small, the viscosity of the bearing lubricant is decreased, on the other hand, the bearing stiffness is extremely decreased under a high-temperature environment. In particular, in a disk drive device provided with a magnetic disk, etc., having a large inertia and a large mass, a large power is applied to a shaft thereof when the device receives an acceleration such as a vibration or an impact, and hence a metal contact between the shaft and the bearing easily occurs when the bearing stiffness is decreased. As a result, a malfunction occurs in the disk drive device.

On the other hand, there has been a problem that, when the molecular weight of a base oil is increased, the bearing stiffness is unnecessarily increased under a low-temperature environment due to an increase in the viscosity of a bearing lubricant. In particular, in the disk drive device provided with the aforementioned magnetic disk, etc., a large drive current flows in order to activate the device, and hence a power consumption is increased due to an increase in a load under a low-temperature environment. As a result, in a battery-driven apparatus, etc., the life span thereof is extremely shortened.

Due to such problems, a disk drive device has been currently limited in being mounted in a mobile apparatus possibly used in a wide temperature range, such as a mobile apparatus used outdoors.

It can be considered that the fluidity of a bearing lubricant to be used is secured in a wide temperature range in order to improve the performance of a bearing. In order to meet the aforementioned demands for high-performance of an electronic device, it is expected, for example, that a bearing lubricant, the pour point of which is −60° C. or less, is needed.

### SUMMARY OF THE INVENTION

The present invention has been made based on the aforementioned acknowledgments by the inventor, and one of the purposes thereof is to provide a technique in which the performance of a bearing can be further improved.

An embodiment of the present invention relates to a bearing lubricant. The bearing lubricant includes a base oil containing an ester compound consisting of 2-ethyl-2-methyl-1,3-propanediol and at least one of carboxylic acids having 5 to 12 carbon atoms, the ester compound being represented by the following formula (1), in which the base oil has a kinetic viscosity at approximately 40° C. of approximately 7 to 15 mPa, and has a pour point of approximately −60° C. or less:

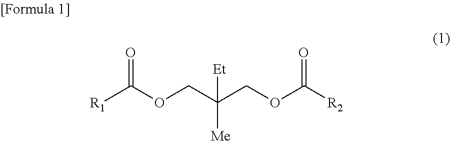

where R1 and R2 represent hydrocarbon groups having 4 to 11 carbon atoms.

It is noted that any combination of the aforementioned components or any manifestation of the present invention exchanged between methods, devices systems and so forth, is effective as an embodiment of the present invention.

### BRIEF DESCRIPTION OF THE DRAWINGS

An embodiment will now be described, by way of example only, with reference to the accompanying drawing which are meant to be exemplary, not limiting, in which:

FIG. 1 is a schematic cross-sectional view illustrating the structure of a disk drive device according to an embodiment 1.

### DETAILED DESCRIPTION OF THE INVENTION

The invention will now be described by reference to the preferred embodiments. This does not intend to limit the scope of the present invention, but to exemplify the invention.

The present invention will now be described based on a preferred embodiment. The preferred embodiment does not intend to limit the scope of the invention but exemplify the invention. All of the features and the combinations thereof described in the embodiment are not necessarily essential to the invention. It can be readily conceived by those skilled in the art that various variations may be made by making various combinations of the aforementioned components, which are also encompassed in the scope of the present invention.

### Embodiment

A bearing lubricant according to the present embodiment is preferably used in a bearing such as a fluid dynamic bearing, etc. Hereinafter, the composition of the bearing lubricant according to the embodiment will be described in detail.

The bearing lubricant according to the present embodiment includes a base oil containing an ester compound represented by the following formula (1):

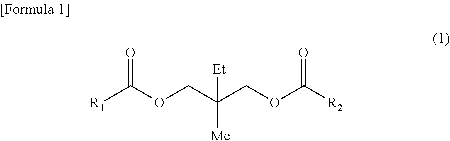

where R1 and R2 represent hydrocarbon groups having 4 to 11 carbon atoms.

That is, the base oil contains, as its main body, an aliphatic diol ester consisting of 2-ethyl-2-methyl-1,3-propanediol as a diol and at least one of carboxylic acids having 5 to 12 carbon atoms. The base oil may consist of only an aliphatic diol ester. In the formula (1), R1 and R2 derived from the aforementioned carboxylic acids are hydrocarbon groups having 4 to 11 carbon atoms, and the number of the carbon atoms is preferably 7 to 10, and more preferably 7 or 8. The hydrocarbon groups are saturated or unsaturated aliphatic hydrocarbons having linear, branched or cyclic structures. R1 and R2 may or may not be identical to each other.

Herein, when the hydrocarbon groups R1 and R2 have branched structures, the pour point of the bearing lubricant can generally be lowered, but the viscosity thereof is sometimes increased. On the other hand, when the hydrocarbon groups R1 and R2 have linear structures, the viscosity thereof is not generally increased, but it is difficult to lower the pour point thereof. In contrast, in particular, the pour point of the bearing lubricant can be more preferably lowered without increasing the viscosity thereof, by using saturated linear aliphatic carboxylic acids having 8 or 9 carbon atoms. Accordingly, the carboxylic acid contained in the aforementioned ester compound is preferably a saturated linear aliphatic carboxylic acid having 8 or 9 carbon atoms, more preferably a saturated linear aliphatic carboxylic acid having 9 carbon atoms. Generally, with a decrease in the number of the carbon atoms in the carboxylic acid, the kinetic viscosity thereof is decreased and an evaporation loss is increased; on the other hand, with an increase in the number thereof, the kinetic viscosity is increased and an evaporation loss is decreased.

The diol of the ester compound is 2-ethyl-2-methyl-1,3-propanediol. A methyl group and an ethyl group are bonded to the carbon atom in the second position of the 1,3-propanediol, and hence the ester compound can contain an enantiomer. Thereby, the pour point of the bearing lubricant can be lowered. Also, the freezing point thereof can be lowered. In the case of the structure in which a methyl group and an ethyl group are bonded to the carbon atom in the second position (hereinafter, sometimes referred to as ethyl methyl), the pour point of the bearing lubricant can be lowered while the viscosity index thereof is being relatively high, in comparison with the case of the structure in which a propyl group and a methyl group are bonded thereto (hereinafter, sometimes referred to as propyl methyl). On the other hand, in the case of the structure in which two methyl groups are bonded thereto (hereinafter, sometimes referred to as dimethyl), the pour point becomes high although the viscosity index is approximately the same as the ethyl methyl, i.e., relatively high in comparison with the propyl methyl. Further, in the case of the propyl methyl, the pour point can be lowered, but the viscosity index becomes low in comparison with the ethyl methyl and dimethyl.

The kinetic viscosity at approximately 40° C. of the base oil is approximately 5 to 20 mPa, preferably approximately 7 to 15 mPa. The base oil having the aforementioned kinetic viscosity can be preferably used in a bearing such as a fluid dynamic bearing. Alternatively, the kinetic viscosity at approximately 40° C. of the aliphatic acid diol ester represented by the formula (1) can be approximately 5 to 20 mPa, preferably approximately 7 to 15 mPa. Further, the pour point of the base oil is approximately −40° C. or less, preferably approximately −60° C. or less, and hence the bearing can rotatably support a rotating body even under a low-temperature condition.

The bearing lubricant according to the present embodiment may include an antioxidant containing at least one of a hindered phenolic antioxidant and a hindered amine antioxidant. The antioxidant is contained in the bearing lubricant in an amount of, for example, approximately 0.05 wt % to approximately 10.0 wt % based on the total weight of the bearing lubricant. For example, the antioxidant is contained in the bearing lubricant in amount of approximately 0.1 wt % or more based on the total weight of the bearing lubricant.

Examples of the hindered phenolic antioxidant contain, for example: a monophenol antioxidant such as 2,6-di-tert-butyl-4-hydroxytoluene and n-octadecyl-3-(3′,5′-di-tert-butyl-4′-hydroxyphenyl)propionate; a diphenol antioxidant such as 4,4′-butylidene bis(3-methyl-6-tert-butylphenol) and 4,4′-methylene bis (4-methyl-6-tert-butylphenol); and a phenol antioxidant having three or more of 2,6-di-tert-butyl-4-hydroxy structures. These phenolic antioxidants may be used alone or in combination as a mixture of two or more types. The hindered phenolic antioxidant is preferably a phenolic antioxidant having three or more of 2,6-di-tert-butyl-4-hydroxy structures; and is preferably contained in the bearing lubricant in an amount of approximately 0 to 10.0 wt % (i.e., 10.0 wt % or less) based on the total weight of the bearing lubricant.

Examples of the hindered amine antioxidant contain, for example: dialkyldiphenylamine, dioctyldiphenylamine and 4,4′-bis(α,α-dimethylbenzyl)diphenylamine. These amine antioxidants may be used alone or in combination as a mixture of two or more types. The hindered amine antioxidant is preferably a dioctyldiphenylamine or 4,4′-bis(α,α-dimethylbenzyl)diphenylamine; and is preferably contained in the bearing lubricant in an amount of approximately 0 to 10.0 wt % (i.e., 10.0 wt % or less) based on the total weight of the bearing lubricant.

The bearing lubricant according to the present embodiment may contain at least one of a phosphate ester and a phosphite ester. In the present embodiment, the phosphate ester and the phosphite ester is added as a wear adjuster.

Examples of the phosphate ester as a wear adjuster, i.e., a phosphate ester wear adjuster contain, for example: a trialkyl phosphate ester such as tributyl phosphate and trioctyl phosphate; and a triaryl phosphate such as triphenyl phosphate, tricresyl phosphate and tris(nonylphenyl)phosphate. Such a phosphate ester wear adjustment is preferably contained in the bearing lubricant in an amount of approximately 0.01 to 5.0 wt % based on the total weight of the bearing lubricant.

Examples of the phosphite ester as a wear adjuster, i.e., a phosphite ester wear adjuster contain, for example: an alkoxide phosphite ester such as tributyl phosphite and trioctyl phosphite; a phenoxide-type phosphite ester such as triphenyl phosphite and tri(octylphenyl)phosphite; and a hybrid type of these phosphite esters. Such a phosphite ester wear adjuster is preferably contained in the bearing lubricant in an amount of approximately 0.01 to 5 wt % based on the total weight of the bearing lubricant.

Further, the bearing lubricant according to the present embodiment may contain a phosphorus extreme-pressure additive.

Further, in the bearing lubricant according to the present embodiment, the base oil may contain the ester compound represented by the formula (1) in an amount of approximately 40 wt % or more based on the total weight of the bearing lubricant, and contain a poly-α-olefin having a kinetic viscosity of approximately 5 mPa or more.

Further, the base oil may contain the ester compound represented by the formula (1) in an amount of approximately 40 wt % or more based on the total weight of the bearing lubricant, and contain a diester having a kinetic viscosity of approximately 6 mPa or more.

Further, the base oil may contain the ester compound represented by the formula (1) in an amount of approximately 40 wt % or more, and contain a trimethylpropane ester having a kinetic viscosity of approximately 6 mPa or more.

The bearing lubricant according to the present embodiment is preferably used in a bearing rotatably supporting a rotating body. When using the bearing lubricant according to the embodiment in the bearing, the resistance between the rotating body and the bearing can be kept small for a longer period and under a lower-temperature environment.

To sum up the operation and effect of the aforementioned bearing lubricant according to the present embodiment, the bearing lubricant according thereto includes a base oil containing the ester compound represented by the aforementioned formula (1), the ester compound consisting of 2-ethyl-2-methyl propanediol and at lest one of carboxylic acids having 5 to 12 carbon atoms, in which the base oil has a kinetic viscosity at approximately 40° C. of approximately 7 to 15 mPa, and has a pour point of approximately −60° C. or less. With this, a bearing lubricant having a low viscosity and low volatility can be obtained, allowing an evaporation loss of the bearing lubricant to be reduced, and allowing an increase in the viscosity of the rearing lubricant to be suppressed. Further, a bearing lubricant, the pour point of which is low and a viscosity change of which is small, can be obtained. As a result, an energy loss in the bearing can be suppressed for a long period. Further, the bearing can stably support a rotating body rotatably under a low-temperature environment. As a result, a motor mounted with the bearing, and an electronic apparatus mounted with the motor, can be less power consuming, can have longer life-spans, and can be used in enlarged temperature ranges.

For example, when a fluid dynamic bearing using the bearing lubricant according to the present embodiment is mounted in a disk drive device, the disk drive device can be used for a long period due to the improvement in the evaporation loss of the bearing lubricant. Accordingly, the frequency of replacement of the disk drive device is reduced, allowing the situations in which many components have to be discarded because of only a single bearing, to be reduced. As a result, wasteful consumption of the limited resources can be reduced.

On the other hand, because the pour point of the bearing lubricant is lowered to a lower temperature, the bearing stiffness is not unnecessarily increased under a low-temperature environment, allowing an increase in the consumption current due to an increase in the load, to be prevented. Accordingly, even when an apparatus mounted with the disk drive device is battery-driven, the apparatus can be used for an extended period. As a result, the disk drive device can be preferably mounted in mobile apparatuses used outdoors.

For example, according to the present invention, a hard disk drive device, and a spindle motor and a polygon mirror scanner motor, etc., which can be applied to the hard disk drive device, can be improved in their performance.

When the carboxylic acid is a saturated linear aliphatic carboxylic acid having 8 or 9 carbon atoms, the pour point of the bearing lubricant can be lowered more preferably, without increasing the viscosity of the bearing lubricant. Further, by containing an antioxidant containing at least one of the hindered phenolic antioxidant and the hindered amine antioxidant, oxidization of the bearing lubricant can be prevented, thereby allowing the bearing lubricant to be used for a further longer period. When the antioxidant is contained in an amount of approximately 0.05 wt % to approximately 10.0 wt % based on the total weight of the bearing lubricant, the characteristics of the bearing lubricant such as the low viscosity, low volatility and low pour point, etc., can be less affected. Further, when at least one of the phosphate ester and the phosphite ester is contained in the bearing lubricant, damages due to the wear between the bear and the rotating body can be reduced, allowing the bearing to be used for a longer period.

### (Structures of Bearing and Disk Drive Device)

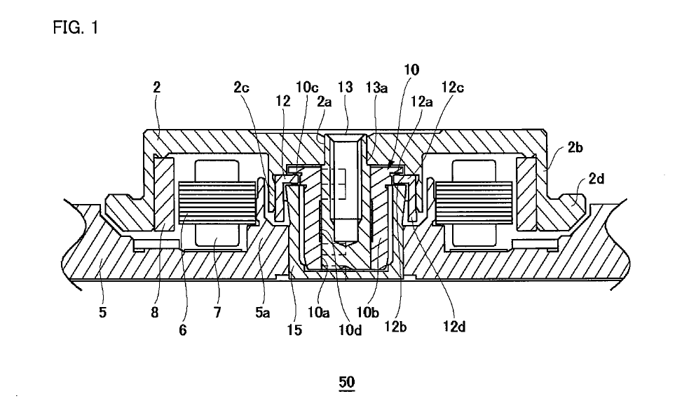

Subsequently, the structures of a bearing in which the bearing lubricant according to the present embodiment can be used, and a disk drive device provided with the bearing, will be described. FIG. 1 is a schematic cross-sectional view illustrating the structure of a disk drive device according to an embodiment 1. In the following descriptions, for convenience, the lower portion indicated in the view is represented as the bottom, and the upper portion as the top.

The disk drive device 50 configured to drive a hard disk comprises a fixed body, a radial fluid dynamic bearing, a thrust fluid dynamic bearing and a rotating body. A rotating speed of the rotating body is, for example, 5400 rpm.

The fixed body is configured to include: a base member 5; a stator core 6 fixed to the outer circumferential surface of a cylindrical portion 5a provided in the base member 5; a circular housing member 15 fixed to the inner circumferential surface of the cylindrical portion 5a; and a circular shaft housing member 10 having the inner circumferential surface 10a of the cylindrical portion, which is fixed to the inner circumferential surface of the housing member 15.

A plurality of salient poles (not illustrated) extending outwards of the stator core 6 are wounded with coils 7. The shaft housing member 10 has a cylindrical portion 10b that has the inner circumferential surface 10a of the cylindrical portion and that houses a shaft, and a flange portion 10c that is connected to one end side of the cylindrical portion 10b and that extends outwards of the cylindrical portion 10b. The housing member 15 has an approximately cup-like shape including: a cylindrical portion, to the inner circumference of which the shaft housing member 10 is fitted; a bottom portion that seals one end of the cylindrical portion; and an upper-end surface portion that is provided at the other end of the cylindrical portion and that has a surface in the axial direction.

The rotating body is configured to include an approximately cup-shaped hub 2, a shaft 13 fixed to a central hole 2a of the hub 2, a ring-shaped magnet 8 and a thrust member 12. The hub 2 is configured to include a first cylindrical portion 2c that is concentric with the central hole 2a and has a small diameter, a second cylindrical portion 2b provided outward of the first cylindrical portion 2c, and a hub outward extension portion 2d that extends outwards at the lower end of the second cylindrical portion 2b. The thrust member 12 is fixed to the inner circumferential surface of the first cylindrical portion 2c, and the ring-shaped magnet 8 is fixed to the inner circumferential surface of the second cylindrical portion 2b.

A shoulder portion 13a is provided in the upper end portion of the shaft 13, and the diameter of the shaft 13 lower than the shoulder portion 13a is slightly larger than that of the central hole 2a of the hub 2, and the diameter of the shaft 13 upper than the shoulder portion 13a is approximately the same as that of the central hole 2a. The shaft 13 and the hub 2 are connected together by the shaft 13 being press-fitted into the central hole 2a of the hub 2, and by the outer circumferential surface of the shaft 13 upper than the shoulder portion 13a being in contact with the inner circumferential surface of the central hole 2a, and by the shoulder portion 13a being engaged with the hub 2 at the lower end of the central hole 2a.

The thrust member 12 includes: a disk portion 12c that has a thrust upper surface 12a and a thrust lower surface 12b, and that is thin in the axial direction; and a descender portion 12d that is combined to the disk portion 12c on the lower surface on the outer circumferential side of the disk portion 12c and that is long in the axial direction. The inner circumferential surface of the descender portion 12d has a tapered-shape in which the radius thereof is gradually becomes smaller toward the tip thereof. In cooperation with the outer circumferential surface of the housing member 15, the inner circumferential surface of the descender portion 12d forms a capillary seal portion that prevents a lubricant filled in a gap of the dynamic bearing, from leaking outside by capillarity.

The disk portion 12c of the thrust member 12 is arranged between the lower surface of the flange portion 10c of the shaft housing member 10 and the upper end surface of the housing member 15 so as to create a narrow gap with each of the surfaces. The outer circumference of the descender portion 12d is fixed to the inner circumferential surface of the first cylindrical portion 2c of the hub 2. The thrust fluid dynamic bearing is formed by providing thrust dynamic pressure grooves (not illustrated) on both surfaces of the thrust upper surface 12a and the thrust lower surface 12b.

The radial fluid dynamic bearing is configured to include: the shaft 13; the shaft housing member 10 that houses the shaft 13 and supports it rotatably; a first and a second herringborn-shaped radial dynamic pressure grooves (not illustrated) that are arranged so as to be spaced apart from each other in the axial direction, on the inner circumferential surface 10a of the cylindrical portion of the shaft housing member 10; a circumferential concave portion 10d that is arranged in the intermediate portion between the first and the second radial dynamic pressure grooves; and a bearing lubricant that is filled in a gap between the outer circumferential surface of the shaft 13 and the inner circumferential surface 10a of the cylindrical portion of the shaft housing member 10.

In the present embodiment, the shaft housing member 10 is formed of a copper material. And, by using the aforementioned fluid dynamic bearing, the shaft housing member 10 is designed to have an inner diameter of the cylindrical portion 10b of approximately 2.5 mm and a thickness of the cylindrical portion 10b where the dynamic pressure groove is formed, of approximately 0.6 mm. And, by forming the dynamic pressure groove having a depth of approximately 5 μm on the inner circumferential surface 10a of the cylindrical portion with the use of a predetermined cutting process, a herringborn-shaped dynamic pressure groove is provided. The rotating body is rotatably supported by the radial fluid dynamic bearing and the thrust fluid dynamic bearing, and is rotationally driven by an electromagnetic action between the stator core 6 and the ring-shaped magnet 8.

Further, a magnetic disk (not illustrated) is mounted on the outer circumference of the hub 2, and recording or reading of data is executed by reading/writing means (not illustrated).

When the kinetic viscosities at approximately 40° C. of a conventional bearing lubricant and the bearing lubricant according to the present embodiment, were adjusted at 9.5 cst, a drive current at a low-temperature of approximately 0° C. was approximately 210 mA in a disk drive device using the conventional bearing lubricant, while that on the same conditions was approximately 150 mA in the disk drive device using the bearing lubricant according to the embodiment. From the result, it can be understood that the bearing lubricant according to the embodiment has an effect of reducing a drive current.

### EXAMPLES

Hereinafter, examples of the present invention will be described, which do not intend to limit the scope of the invention, but are presented as preferred illustrative examples of the invention.

### Bearing Lubricant

### Examples 1 and 2 and Comparative Examples 1 and 2

According to components prescribed in the following table 1, base oils of Examples 1 and 2 were produced. The base oil according to Example 1 includes 2-ethyl-2-methyl-1,3-propanediol dioctyl ester, and that according to Example 2 includes 2-ethyl-2-methyl-1,3-propanediol dinonyl ester. A diol ester (DOE) and a neopentyl glycol (NPG) (2,2-dimethylpropanediol) were prepared as Comparative Examples 1 and 2, respectively.

TABLE 1

| | | | COMPARATIVE | COMPARATIVE |
|---|---|---|---|---|
| | EXAMPLE 1 | EXAMPLE 2 | EXAMPLE 1 | EXAMPLE 2 |
| BASE OIL | FORMULA 1 | FORMULA 1 | DOE | NPG |
| | (R1, R2 = C7H15) | (R1, R2 = C8H17) | (DIOL ESTER) | (NEOPENTYL |
| | | | | GLYCOL) |
| KINETIC VISCOSITY (cSt) | 7.68 | 9.09 | 10.41 | 8.49 |
| VISCOSITY CHANGE | 0.78 | 0.68 | 2.40 | 2.71 |
| RATE AFTER | | | | |
| THE TEST (%) | | | | |
| EVAPORATION | −1.25 | −0.28 | −1.42 | −0.45 |
| TEST (WT %) | | | | |
| FLOW POINT (° C.) | <−65 | <−65 | −50 | <−65 |

### (Viscosity Measurement, Evaporation Test, Pour Point Measurement)

Viscosities were measured by using a rotational viscometer. The measurement was performed at approximately 40° C., which is generally adopted. In the evaporation test, an evaporation loss by wt % was calculated from a weight change that was obtained in the following way: each composition of Examples 1 and 2 and Comparative Examples 1 and 2 was placed in a glass beaker; the glass beaker was left in a forced convection constant-temperature oven of approximately 100° C. for 240 hours; and weights before and after the heating were measured. The aforementioned viscosity measurement was performed on each composition after the evaporation test to obtain a viscosity change rate before and after the evaporation test. A pour point was measured by a method according to JIS K2269.

As a result, it has been learned that the base oils of Embodiments 1 and 2 have low viscosities in comparison with Comparative Example 1, and have higher properties in the evaporation loss, viscosity change rate and pour point. Further, the base oils of Examples 1 and 2 have extremely lower viscosity change rates than that of Comparative Example 2.

What is claimed is:

1. A bearing lubricant including a base oil containing an ester compound consisting of 2-ethyl-2-methyl-1,3-propanediol and at least one of carboxylic acids having 5 to 12 carbon atoms, the ester compound being represented by the following formula (1), wherein the base oil has a kinetic viscosity at approximately 40° C. of approximately 7 to 15 mPa, and has a pour point of approximately −60° C. or less: 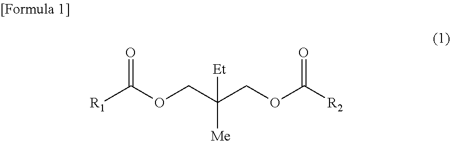
where R1 and R2 represent hydrocarbon groups having 4 to 11 carbon atoms.

2. The bearing lubricant according to claim 1, wherein the carboxylic acid is a saturated linear aliphatic carboxylic acid having 8 or 9 carbon atoms.

3. The bearing lubricant according to claim 1 including an antioxidant containing at least one of a hindered phenolic antioxidant and a hindered amine antioxidant.

4. The bearing lubricant according to claim 3 containing the antioxidant in an amount of approximately 0.05 wt % to approximately 10.0 wt % based on the total weight of the bearing lubricant.

5. The bearing lubricant according to claim 1 containing at least one of a phosphate ester and a phosphite ester.

6. The bearing lubricant according to claim 5 containing at least one of the phosphate ester and the phosphite ester in an amount of approximately 0.01 to 5.0 wt % based on the total weight of the bearing lubricant.

7. A bearing rotatably supporting a rotating body configured to use a bearing lubricant including a base oil containing an ester compound consisting of 2-ethyl-2-methyl-1,3-propanediol and at least one of carboxylic acids having 5 to 12 carbon atoms, the ester compound being represented by the following formula (1), wherein the base oil has a kinetic viscosity at approximately 40° C. of approximately 7 to 15 mPa, and has a pour point of approximately −60° C. or less: 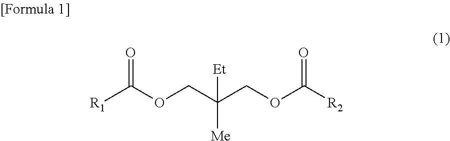
where R1 and R2 represent hydrocarbon groups having 4 to 11 carbon atoms.

8. The bearing according to claim 7, wherein, in the bearing lubricant, the carboxylic acid is a saturated linear aliphatic carboxylic acid having 8 or 9 carbon atoms.

9. The bearing according to claim 7, wherein the bearing lubricant includes an antioxidant containing at least one of a hindered phenolic antioxidant and a hindered amine antioxidant.

10. The bearing according to claim 9, wherein the bearing lubricant contains the antioxidant in an amount of approximately 0.05 wt % to approximately 10.0 wt % based on the total weight of the bearing lubricant.

11. The bearing according to claim 7, wherein the bearing lubricant contains at least one of a phosphate ester and a phosphite ester.

12. The bearing according to claim 11, wherein the bearing lubricant contains at least one of the phosphate ester and the phosphite ester in an amount of approximately 0.01 to 5.0 wt % based on the total weight of the bearing lubricant.

13. A disk drive device comprising a bearing configured to use a bearing lubricant including a base oil containing an ester compound consisting of 2-ethyl-2-methyl-1,3-propanediol and at least one of carboxylic acids having 5 to 12 carbon atoms, the ester compound being represented by the following formula (1), wherein the base oil has a kinetic viscosity at approximately 40° C. of approximately 7 to 15 mPa, and has a pour point of approximately −60° C. or less: 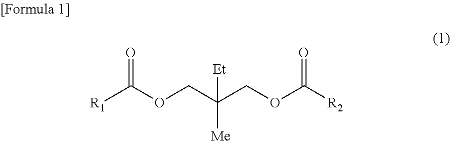
where R1 and R2 represent hydrocarbon groups having 4 to 11 carbon atoms.

14. The disk drive device according to claim 13, wherein the bearing comprises at least one of a radial fluid dynamic bearing and a thrust fluid dynamic bearing.

15. The disk drive device according to claim 13, wherein, in the bearing lubricant, the carboxylic acid is a saturated linear aliphatic carboxylic acid having 8 or 9 carbon atoms.

16. The disk drive device according to claim 13, wherein the bearing lubricant includes an antioxidant containing at least one of a hindered phenolic antioxidant and a hindered amine antioxidant.

17. The disk drive device according to claim 16, wherein the bearing lubricant contains the antioxidant in an amount of approximately 0.05 wt % to approximately 10.0 wt % based on the total weight of the bearing lubricant.

18. The disk drive device according to claim 13, wherein the bearing lubricant contains at least one of a phosphate ester and a phosphite ester.

19. The disk drive device according to claim 18, wherein the bearing lubricant contains at least one of the phosphate ester and the phosphite ester in an amount of approximately 0.01 to 5.0 wt % based on the total weight of the bearing lubricant.

20. The disk drive device according to claim 13 configured to be mounted in a battery-driven mobile apparatus.
